(19) United States
(12) Patent Application Publication
(10) Pub. No.: US 2026/0272529 A1
(43) Pub. Date: Sep. 17, 2026

(54) COUPLING STRUCTURE FOR AN INTRAMEDULLARY NAIL AND INSERTION DEVICE

(71) Applicant: JEIL MEDICAL CORPORATION, Seoul (KR)

(72) Inventors: Dong Kyu LEE, Bucheon-si (KR); Chi-Hoon Kim, Goyang-si (KR); Seon-Yong Park, Seoul (KR)

(21) Appl. No.: 19/561,413

(22) Filed: Mar. 9, 2026

(30) Foreign Application Priority Data
Mar. 12, 2025 (KR) 10-2025-0031937

(51) Int. Cl. A61B 17/88; A61B 17/00; A61B 17/72

(52) U.S. Cl. CPC A61B 17/8872; A61B 17/72; A61B 2017/00477

(57) ABSTRACT

A coupling structure of an intramedullary nail and an insertion device including an intramedullary nail configured to be inserted into a medullary canal, and an insertion device configured to insert the intramedullary nail into the medullary canal, in which the intramedullary nail is configured to be slidably coupled to the insertion device, and the insertion device includes a fixing body configured to be inserted into the intramedullary nail.

### CROSS REFERENCE TO RELATED APPLICATION

This application claims priority from and the benefit of Korean Patent Application No. 10-2025-0031937, filed on Mar. 12, 2025, which is hereby incorporated by reference for all purposes as if fully set forth herein.

### BACKGROUND

### Field

Embodiments of the invention relate generally to an intramedullary nail and an insertion device, and more particularly, to a coupling structure configured to maintain a stable connection between an intramedullary nail and an insertion device during surgery.

### Discussion of the Background

In fracture treatment, an intramedullary nail is widely used as a representative internal fixation device for stably fixing fractures of long bones (such as the femur, tibia, and humerus) and assisting in rapid patient recovery. The intramedullary nail is a medical instrument designed to be inserted into the medullary canal of a bone to stabilize the fracture site and withstand loads during rehabilitation. To accurately insert such an intramedullary nail into the bone, an insertion device is required. The insertion device enables efficiently performing directional control, positional alignment, and force transmission of the intramedullary nail during the surgical procedure.

The coupling structure between the intramedullary nail and the insertion device is a critical element that must guarantee the stability of manipulation and coupling strength during surgery. Accordingly, the insertion device should be firmly coupled to the intramedullary nail. If play or instability occurs at the coupling site, not only accurately inserting the intramedullary nail into the medullary canal becomes difficult, but also unexpected malfunctions may occur during surgery. In particular, the coupling portion between the insertion device and the intramedullary nail should be capable of effectively withstand strong impact forces or rotational forces applied during the process of inserting the intramedullary nail.

However, conventional intramedullary nails have exhibited various problems, such as detachment caused by loosening of the coupling force during surgery or prolonged surgical time due to the occurrence of play at the coupling part.

Accordingly, there is a need for a coupling structure between an intramedullary nail and an insertion device that provides a more stable and efficient connection.

The above information disclosed in this Background section is only for understanding of the background of the inventive concepts, and, therefore, it may contain information that does not constitute prior art.

### SUMMARY

An intramedullary nail and an insertion device according to embodiments of the invention are capable of maintaining a stable coupled state between the intramedullary nails and the insertion device during surgery.

Additional features of the inventive concepts will be set forth in the description which follows, and in part will be apparent from the description, or may be learned by practice of the inventive concepts.

According to an embodiment, a coupling structure includes; an intramedullary nail configured to be inserted into a medullary canal; and an insertion device configured to insert the intramedullary nail into the medullary canal; wherein the intramedullary nail is configured to be primarily coupled by sliding inward from one side of the insertion device, and wherein the insertion device includes a fixing body configured to be inserted into the intramedullary nail to secondarily couple

The intramedullary nail may include: a rod part configured to be inserted into the medullary canal and extending along a longitudinal direction of a bone; and a rod coupling part provided at an end of the rod and configured to be coupled with the insertion device.

The insertion device may include: an insertion coupling part coupled with the rod coupling part and having the fixing body; and an operating part provided at an upper part of the insertion coupling part and configured to insert the fixing body into the rod coupling part.

The insertion device may further include: a handle part extending in one direction from the insertion coupling part to form a grip.

The intramedullary nail may further include: a sliding body provided at the end of the rod part; an inclined surface formed on an upper surface of the sliding body and sloped downward toward an end thereof; sliding grooves formed on both side surfaces of the sliding body; and an insertion hole formed on the inclined surface such that an upper part and a lower part of the sliding body communicate with each other.

The insertion coupling part may include: an upper body forming a main structure; a lower body extending from an end of the upper body; a pair of walls extending downward from both side surfaces of a lower end of the lower body; and a pair of protruding inserts formed to protrude from inner surfaces of the pair of walls and configured to be insertable into the sliding grooves.

The intramedullary nail may further include: an end groove formed at an end of the sliding body, and wherein the insertion coupling part further comprises a stopper formed at a lower part of the lower body and configured to be inserted into the end groove when the protruding inserts are fully inserted into the sliding grooves, thereby limiting further insertion of the sliding body.

The insertion coupling part may further include: a shaft extending through centers of the upper body and the lower body; and wherein the fixing body is formed at an end of the shaft and configured to be inserted into the insertion hole.

The intramedullary nails may further include an internal thread formed inside the insertion hole, and the fixing body may have a thread formed on an outer circumferential surface thereof to engage with the internal thread such that the fixing body and the insertion hole are coupled by screw engagement.

The shaft may be connected to the operating part, and the fixing body may be configured to be inserted into and fixed to the insertion hole by the operating part.

The stopper may have a circular shape, and the stopper may have a diameter less than a diameter of the fixing body.

The sliding grooves may extend in a direction parallel to a slope of the inclined surface.

The pair of protruding inserts may protrude from the inner surfaces of the walls in a direction crossing a longitudinal direction of the upper body.

The stopper may be disposed between the pair of walls along a circumference of the lower part of the lower body.

The upper body of the insertion coupling part may include a plurality of windows through which the shaft is exposed.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a schematic view of a coupling structure between an intramedullary nail and an insertion device according to an embodiment of the invention.

FIG. 2 is an enlarged view of the coupling structure between the intramedullary nail and the insertion device according to an embodiment of the invention.

FIGS. 3, 4 and 5 are perspective views of the intramedullary nail according to an embodiment of the invention.

FIGS. 6, 7, and 8 are perspective views of the insertion device according to an embodiment of the present invention.

FIGS. 9 and 10 are perspective views showing a state before the intramedullary nail slides inward into the insertion device according to an embodiment of the invention.

FIG. 11 is a perspective view showing a state in which the intramedullary nail has slid inward and is inserted into the insertion device according to an embodiment of the invention.

FIG. 12 is a perspective view showing a state in which the fixing body is inserted into the insertion hole and secondarily fixed according to an embodiment of the invention.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

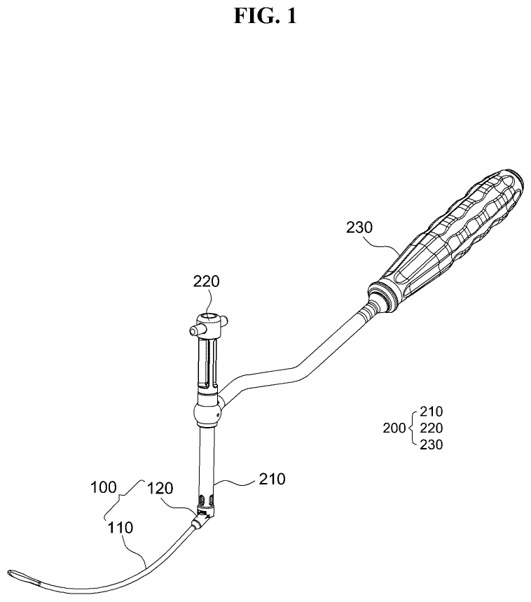

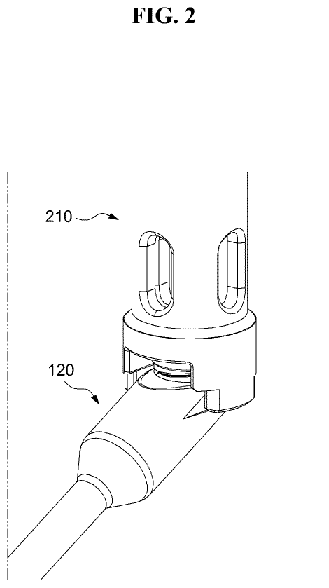

FIG. 1 is a schematic view of a coupling structure between an intramedullary nail and an insertion device according to an embodiment of the invention. FIG. 2 is an enlarged view of the coupling structure between the intramedullary nail and the insertion device.

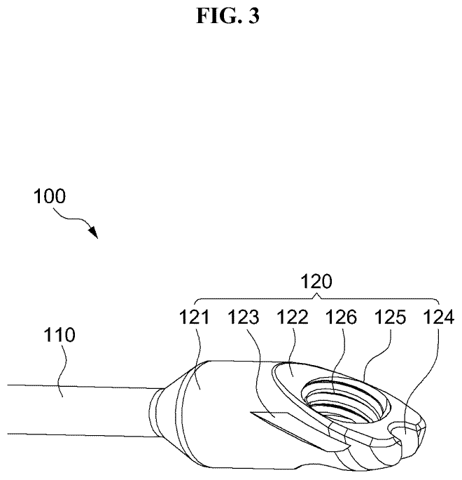

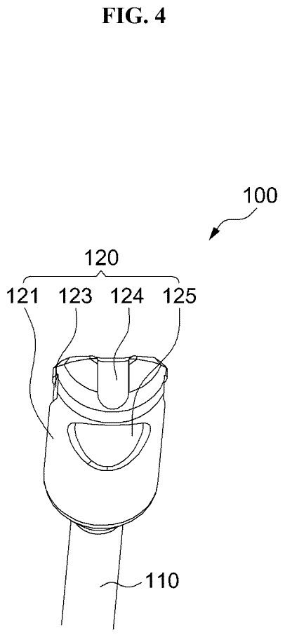

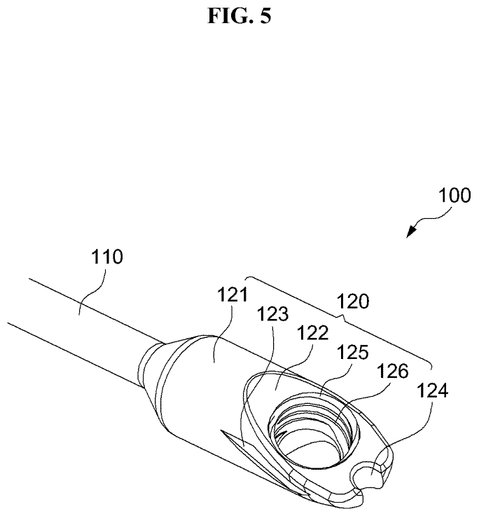

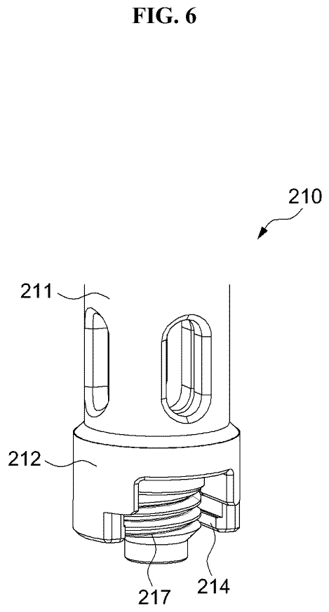

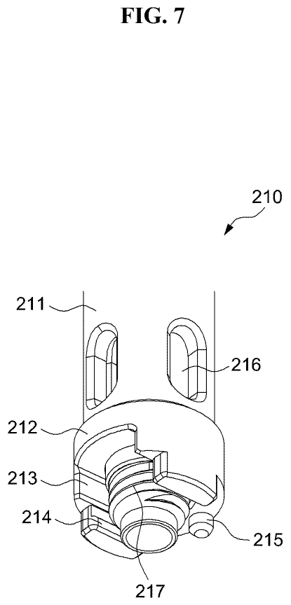

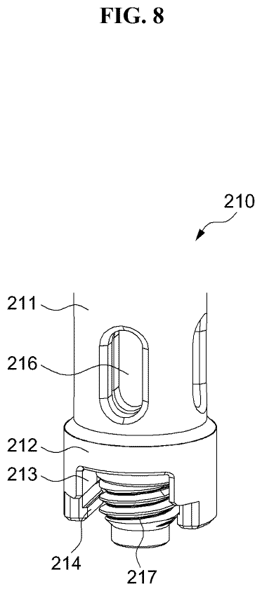

FIGS. 3 to 5 are perspective views of the intramedullary nail according to an embodiment of the invention, and FIGS. 6 to 8 are perspective views of the insertion device.

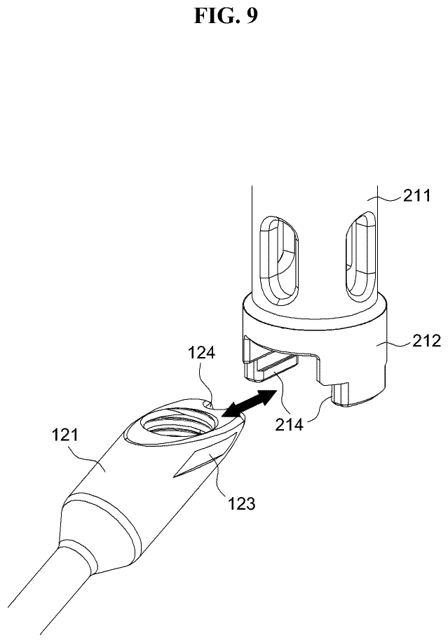

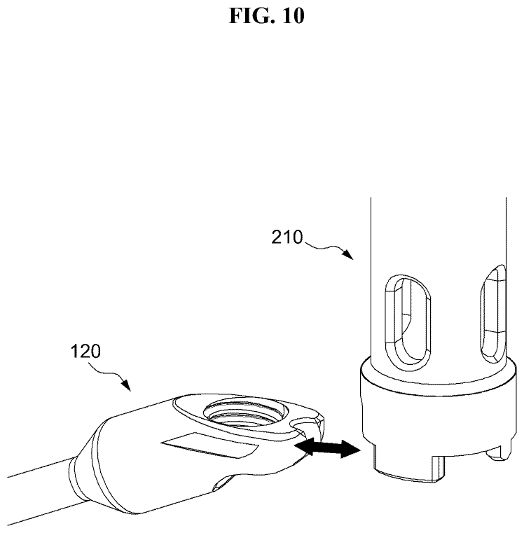

FIGS. 9 and 10 are perspective views showing a state before the intramedullary nail slides inward into the insertion device.

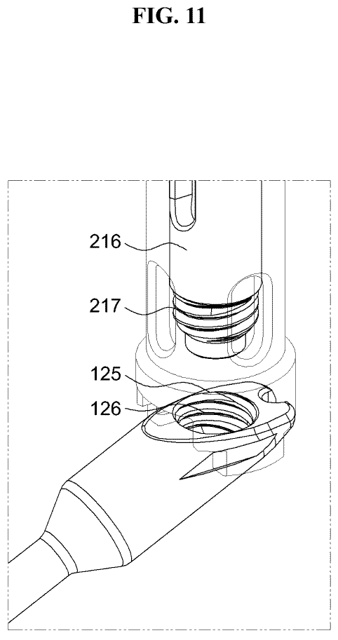

FIG. 11 is a perspective view showing a state in which the intramedullary nail has slid inward and is inserted into the insertion device.

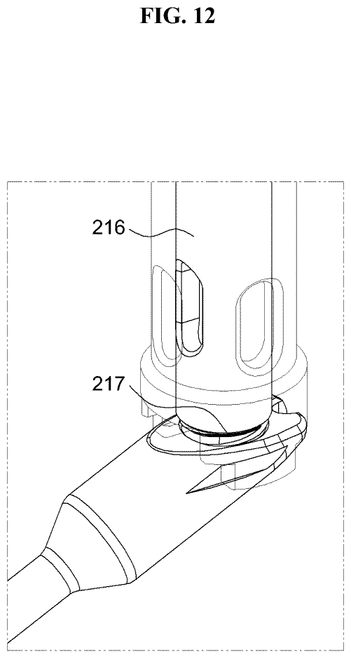

FIG. 12 is a perspective view showing a state in which the fixing body is inserted into the insertion hole and secondarily fixed.

Referring to FIGS. 1 to 12, the intramedullary nail 100 according to an embodiment is configured to be inserted into a medullary canal, and may include a rod part 110 and a rod coupling part 120.

The rod part 110 is configured to be inserted into the medullary canal, and may extend along the longitudinal direction of the bone. The rod part 110 may be made of a biocompatible material. The rod part 110 may be provided to reinforce the internal strength of the bone and disperse the load of the fracture site.

The rod coupling part 120 is provided at an end of the rod part 110 and may be configured to be coupled with the insertion device 200. To this end, the rod coupling part 120 may include a sliding body 121, an inclined surface 122, a sliding groove 123, an end groove 124, an insertion hole 125, and an internal thread 126.

The sliding body 121 is a part formed at the end of the rod part 110. The sliding body 121 may be formed to have a diameter greater than that of the rod part 110, and may form the body of the rod coupling part 120.

The inclined surface 122 is formed on the end side of the upper surface of the sliding body 121 and may be provided to have a downward slope toward the end.

The sliding groove 123 may be formed on a side surface of the sliding body 121. In an embodiment, the sliding groove 123 may be formed on both side surfaces of the sliding body 121. The sliding groove 123 may be formed in the shape of a groove having a certain length. The sliding groove 123 may be formed to extend parallel to the slope of the inclined surface 122.

The end groove 124 is formed at the end of the sliding body 121 and may be provided in the form of a groove into which a stopper 215 of the insertion device 200 can be inserted. The end groove 124 thus engages with the stopper 215 to limit the extent to which the rod coupling part 120 is inserted into the insertion coupling part 210 of the insertion device 200.

The insertion hole 125 is formed on the inclined surface 122 and may be formed as a hole through which the upper and lower portions communicate. The insertion hole 125 may be configured to receive a fixing body 217, to be described later, inserted from above.

The internal thread 126 is a thread formed inside the insertion hole 125 and may be configured to engage with a thread formed on an outer circumferential surface of the fixing body 217.

The insertion device 200 is provided to insert the intramedullary nail 100 into the medullary canal, and may include an insertion coupling part 210, an operating part 220, and a handle part 230.

The insertion coupling part 210 is coupled to the rod coupling part 120 and may include an upper body 211, a lower body 212, a wall 213, a protruding insert 214, a stopper 215, a shaft 216, and a fixing body 217.

The upper body 211 is an upper structure forming the body of the insertion coupling part 210, and may be formed with a cavity capable of accommodating the shaft 216 therein. In an embodiment, the upper body 211 may include a plurality of windows through which the shaft 216 is exposed to the outside.

The lower body 212 extends from the end of the upper body 211 and may be formed to have a greater diameter than the upper body 211.

The walls 213 may extend downward from both side surfaces of the lower end of the lower body 212. More particularly, the lower portion of the lower body 212 may be open at the front and rear (e.g., the direction in which the rod coupling part 120 is inserted) and at the bottom, with only both sides blocked by the walls 213.

The protruding inserts 214 are provided on the inner surfaces of the walls 213, and may protrude toward each other (e.g., in the direction in which the pair of walls 213 face each other or in the direction crossing a longitudinal direction of the upper body 211). In addition, the protruding inserts 214 may be formed to protrude in a shape and length that conforms to the sliding groove 123 so as to slidably inserted into the sliding groove 123.

The inventive concepts are not limited to a particular shape and length of the sliding groove 123 and the protruding inserts 214. In other embodiments, the sliding groove 123 and the protruding inserts 214 may be formed in various shapes, such as points, lines, or planes, with desirable lengths.

In some embodiments, the protruding inserts 214 may be provided in a form where only a part of the wall 213 protrudes, or the entire length of the inner surface of the wall 213 may be provided in a protruding form.

When the protruding insert 214 is inserted into the sliding groove 123, the inclined surface 122 may be provided such that the intramedullary nail 100 is coupled in a tilted state so that the inclined surface 122 is parallel to the bottom surface of the lower body 212.

In this manner, the inclined surface 122 may facilitate insertion of the intramedullary nail 100 into the medullary canal.

When the protruding insert 214 is completely inserted into the sliding groove 123, the stopper 215 is inserted inside the end groove 124 to limit further insertion of the sliding body 121.

To this end, the stopper 215 is provided at the lower portion of the lower body 212 and may be formed to extend downward from the side opposite to the direction in which the sliding body 121 is inserted. At this time, the stopper 215 and the end groove 124 may engage each other in a point contact manner, but may also be engaged to contact each other in a linear or planar form. In an embodiment, the stopper 215 may have a circular shape with a diameter less than a diameter of the fixing body 217, without being limited thereto. In an embodiment, the stopper 215 may be disposed between the pair of walls 213 along a circumference of a lower part of the lower body 212.

The shaft 216 may be formed to extend through the centers of the upper body 211 and the lower body 212.

The fixing body 217 is a structure formed at the lower end of the shaft 216 and may be configured to be inserted into the insertion hole 125 and engaged with the internal thread 126 to be fixed. The fixing body 217 may have a thread formed on its outer circumferential surface that engages with the internal thread 126.

Although the fixing body 217 is exemplarily illustrated as having a screw form, however, the inventive concepts are not limited thereto. In some embodiments, the fixing body 217 may have a structural configuration that enables pin coupling or clamp coupling with the insertion hole 125.

The shaft 216 may be configured to be coupled with operating part 220. More particularly, the operating part 220 is provided at the upper portion of the insertion coupling part 210 and is connected to the shaft 216 to insert the fixing body 217 into the rod coupling part 120. That is, the operating part 220 is connected to the shaft 216, and the fixing body 217 may be inserted into and fixed to the insertion hole 125 by the movement of the operating part 220. For example, when the operating part 220 is rotated by applying pressure thereto, the shaft 216 moves downward, and the thread of the fixing body 217 engages with the internal thread 126 of the insertion hole 125, such that the fixing body 217 is coupled in a state inserted into the insertion hole 125.

The handle part 230 may extend in one direction from the insertion coupling part 210 to form a handle. The handle part 230 may facilitate operation of the insertion device 200, and enable insertion of the intramedullary nail 100.

As described above, the intramedullary nail 100 according to an embodiment is primarily coupled by sliding inward from one side direction of insertion device 200, and the fixing body 217 provided in the insertion device 200 is inserted into the insertion hole 125 of the intramedullary nail 100 to be secondarily coupled, thereby maintaining a stable coupling state between the intramedullary nail 100 and the insertion device 200.

More specifically, according to embodiments of the invention, it is possible to prevent the problem of the intramedullary nail 100 falling off due to weakened coupling force. In addition, because the coupling structure is intuitive, unnecessary movements during surgery may be minimized while maintaining ease of operation. As a result, surgery may be performed more stably, thereby improving the success rate of surgery.

According to the configuration described above, the intramedullary nails is primarily coupled to the insertion device by sliding insertion, and in this state, the fixing body is inserted into the insertion hole to be secondarily coupled. Therefore, a stable coupling state may be maintained.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A coupling structure of an intramedullary nail and an insertion device, comprising:
an intramedullary nail configured to be inserted into a medullary canal; and
an insertion device configured to insert the intramedullary nail into the medullary canal;
wherein the intramedullary nail is configured to be slidably coupled to the insertion device, and
wherein the insertion device includes a fixing body configured to be inserted into the intramedullary nail.

2. The coupling structure of claim 1, wherein the intramedullary nail comprises:
a rod part configured to be inserted into the medullary canal and extending along a longitudinal direction of a bone; and
a rod coupling part provided at an end of the rod part and configured to be coupled with the insertion device.

3. The coupling structure of claim 2, wherein the insertion device comprises:
an insertion coupling part is configured to be coupled with the rod coupling part; and
an operating part provided at an upper part of the insertion coupling part and configured to insert the fixing body into the rod coupling part.

4. The coupling structure of claim 3, wherein the insertion device further comprises a handle part extending in one direction from the insertion coupling part to form a grip.

5. The coupling structure of claim 3, wherein the intramedullary nail further comprises:
a sliding body provided at the end of the rod part;
an inclined surface formed on an upper surface of the sliding body and sloped downward toward an end thereof;
sliding grooves formed on opposing side surfaces of the sliding body; and
an insertion hole formed in the inclined surface and penetrating an upper part and a lower part of the sliding body.

6. The coupling structure of claim 5, wherein the insertion coupling part comprises:
an upper body forming a main structure;
a lower body extending from an end of the upper body;
a pair of walls extending downward from opposing side surfaces of a lower end of the lower body; and
a pair of protruding inserts protruding from inner surfaces of the walls and configured to be insertable into the sliding grooves.

7. The coupling structure of claim 6, wherein:
the intramedullary nail further comprises an end groove formed at an end of the sliding body; and
the insertion coupling part further comprises a stopper formed at a lower part of the lower body and configured to be inserted into the end groove when the protruding inserts are fully inserted into the sliding grooves.

8. The coupling structure of claim 6, wherein:
the insertion coupling part comprises a shaft extending through centers of the upper body and the lower body;
the fixing body is formed at an end of the shaft; and
the fixing body is configured to be inserted into the insertion hole.

9. The coupling structure of claim 8, wherein:
the intramedullary nail further comprises an internal thread formed inside the insertion hole; and
the fixing body includes a thread formed on an outer circumferential surface thereof to engage with the internal thread.

10. The coupling structure of claim 9, wherein:
the shaft is connected to the operating part; and
the fixing body is configured to be inserted into and fixed to the insertion hole by movement of the operating part.

11. The coupling structure of claim 7, wherein:
the stopper has a circular shape; and
the stopper has a diameter less than a diameter of the fixing body.

12. The coupling structure of claim 6, wherein the sliding grooves extend in a direction parallel to a slope of the inclined surface.

13. The coupling structure of claim 6, wherein the pair of protruding inserts protrude from the inner surfaces of the walls in a direction crossing a longitudinal direction of the upper body.

14. The coupling structure of claim 7, wherein the stopper is disposed between the pair of walls along a circumference of the lower part of the lower body.

15. The coupling structure of claim 8, wherein the upper body of the insertion coupling part includes a plurality of windows through which the shaft is exposed.
